(19) United States
(12) Patent Application Publication
(10) Pub. No.: US 2024/0074787 A1
(43) Pub. Date: Mar. 7, 2024

(54) WORKING CHANNEL FOR USE IN A METHOD AND SYSTEM FOR PERCUTANEOUS PROCEDURES

(71) Applicant: Ezriel Kornel, Bedford, NY (US)

(72) Inventor: Ezriel Kornel, Bedford, NY (US)

(21) Appl. No.: 18/274,418

(22) PCT Filed: Jan. 28, 2022
(86) PCT No.: PCT/US2022/014385

§ 371 Date: Jul. 26, 2023

Related U.S. Application Data
(60) Provisional application No. 63/142,725, filed on Jan. 28, 2021.

(51) Int. Cl. A61B 17/34; A61B 17/16; A61B 17/88

(52) U.S. Cl. CPC A61B 17/3417; A61B 17/1659; A61B 17/1671; A61B 17/8897; A61B 2017/00477

(57) ABSTRACT

A working channel for use in a method and system for performing percutaneous procedures extends through the patient's skin and includes a proximal end positioned outside of the patient's body having a first diameter and a distal end positioned inside the user's body and having a second diameter, wherein the second diameter is less than the first diameter.

### CROSS-REFERENCE TO RELATED APPLICATION

The present application claims benefit of and priority to U.S. Provisional Patent Application Ser. No. 63/142,725 filed Jan. 28, 2021 entitled A WORKING CHANNEL FOR USE IN A METHOD AND SYSTEM FOR PERCUTANEOUS PROCEDURES, the entire content of which is incorporated by reference herein.

### BACKGROUND

### Filed of the Disclosure

The present disclosure relates to minimally invasive and percutaneous surgeries and related procedures, such as posterolateral spine fusion, including lumbar spine fusion at the transverse processes, and to medical instruments for performing the same. In particular, the present disclosure relates a method and system of providing minimally invasive percutaneous procedures including a working channel that extends through the patient's skin and includes a proximal end positioned outside of the patient's body having a first diameter and a distal end positioned inside the user's body and having a second diameter, wherein the second diameter is less than the first diameter and including an open side extending from the proximal end to the distal end to allow access to the patient's body along the length of the working channel.

### Related Art

Certain diseases and conditions may require surgical intervention. For example, certain spinal diseases result in pain caused by the movement of vertebrae relative to each other. One treatment for such conditions is a surgical procedure that fuses one or more vertebrae together to limit such movement and thus limit the associated pain. Such surgeries typically require an open incision followed by removal of a portion of bone and application of a bone fusing substance. Such procedures, however, are invasive and limited in application to a particular application site.

Conventional percutaneous procedures utilize tools that typically access a single position in the patient's body. In some procedures, access to and vision of multiple positions in the user's body is required, which requires multiple incisions to allow for repeated repositioning and viewing of multiple positions in the user's body which is invasive and causes trauma to the tissue.

Accordingly, it would be beneficial to provide a method, system and tools for minimally invasive percutaneous procedures that allow for the use of additional equipment to provide for a broad spectrum of potential applications and treatment at multiple sites in the user's body via one incision.

### SUMMARY

In light of the above, the present disclosure relates to a working channel for use in a method and system for performing percutaneous procedures, the working channel includes a proximal end extending outside of a patient's body and having a first diameter and a distal end positioned in the patient's body and having a second diameter, where the first diameter is larger than the second diameter and the proximal end is sized to receive additional treatment or diagnostic equipment outside of the body, such as an endoscope or suction tube, for example, while the distal end with the smaller diameter minimizes disruption to the user's body in introducing these tools to the user's body. The working channel includes at least one open side extending from the proximal end to the distal end to allow for access to the patient's body at multiple locations along the length of the working channel

A system for performing percutaneous procedures in accordance with an embodiment of the present disclosure includes: a working channel, the working channel including: an inner wall; an outer wall surrounding the inner wall; a side opening formed between the inner wall and the outer wall along a length of the working channel; a first end having a first width; a second end having a second width, wherein the second width is less than the first width; and at least one tool configured to be received in the working channel.

In embodiments, the system may include at least one fastener connected to the working channel, wherein the at least one fastener is configured to receive a suction hose connected to a suction source.

In embodiments, the system may include an integral suction element formed in at least one of the inner wall and the outer wall of the working channel.

In embodiments, the inner wall includes at least one suction opening formed therein and in fluid communication with the integral suction element.

In embodiments, the system includes a suction connector provided in at least one of the inner wall and the outer wall and configured to provide a connection between the integral suction element and a suction supply.

In embodiments, the at least one tool comprises a trocar configured to make an incision in a patient's skin and extend into the patient's body.

In embodiments, the working channel is configured to enter the user's skin with the first end positioned under the user's skin and the second end extending outside of the user's skin.

In embodiments, the trocar is configured to be received in the working channel and withdrawn therefrom after the trocar reaches a desired position in the patient's body.

In embodiments, the system includes a second tool configured to be received in the inner cavity of the working channel.

In embodiments, the second tool includes a dilator configured to expand a path made by the trocar through the user's body and configured to be removed from the working channel after extending to the point of interest in the user's body.

In embodiments, the second tool is an endoscope.

In embodiments, the second tool is a syringe.

In embodiments, the second tool is a decorticator.

A working channel in accordance with an embodiment of the present disclosure includes an inner wall; an outer wall surrounding the inner wall; a side opening formed between the inner wall and the outer wall along a length of the working channel; a first end having a first width; and a second end having a second width, wherein the second width is less than the first width.

In embodiments, the working channel includes at least one fastener connected to the working channel, wherein the at least one fastener is configured to receive a suction hose connected to a suction source.

In embodiments, the working channel includes an integral suction element formed in at least one of the inner wall and the outer wall.

In embodiments, the working channel includes at least one suction opening formed in the inner wall and in fluid communication with the integral suction element.

In embodiments, the working channel includes a plurality of suction openings formed in the inner wall and in fluid communication with the integral suction element.

In embodiments, the working channel includes at least one suction connector configured to provide a fluid connection between the integral suction element and a suction supply.

In embodiments, the working channel includes a second integral suction element formed in a least one of the inner wall and the outer wall.

### BRIEF DESCRIPTION OF THE DRAWINGS

Exemplary embodiments of the present disclosure will be described with reference to the accompanying figures, wherein:

FIG. 1 illustrates a schematic representation of two vertebrae with an exemplary dilator positioned relative to the transverse processes of the two vertebrae in accordance with an embodiment of the present disclosure;

FIG. 2 illustrates a dilator positioned relative to the transverse processes of three vertebrae from a side view in accordance with an embodiment of the present disclosure;

FIG. 3 illustrates an exemplary schematic of a single vertebra;

FIG. 4 illustrates an exemplary set of tools including a working channel suitable for use in the method and system of providing a minimally invasive percutaneous procedure in accordance with an embodiment of the present disclosure;

FIG. 5A illustrates a detailed view an exemplary embodiment of the working channel used in the method and system of providing minimally invasive percutaneous procedures in accordance with an embodiment of the present disclosure;

FIG. 5B illustrates a cross-sectional view of a distal end of the working channel of FIG. 5A in accordance with an embodiment of the present disclosure;

FIG. 5C illustrates a cross-sectional view of a proximal end of the working channel of FIG. 5B in accordance with an embodiment of the present disclosure;

FIG. 6A illustrates a detailed view of an exemplary embodiment of a working channel used in the method and system of providing minimally invasive percutaneous procedures in accordance with another embodiment of the present disclosure;

FIG. 6B illustrates a cross-sectional view of a distal end of the working channel of FIG. 6A in accordance with an embodiment of the present disclosure;

FIG. 6C illustrates a cross-sectional view of a proximal end the working channel of FIG. 6A in accordance with an embodiment of the present disclosure;

FIG. 7 illustrates an exemplary view of the working channel of FIGS. 6A, 6B and 6C including a suction tube secured thereto;

FIG. 8A illustrates a detailed view of an exemplary embodiment of a working channel used in the method and system of providing minimally invasive percutaneous procedures in accordance with another embodiment of the present disclosure;

FIG. 8B illustrates a cross-sectional view of a distal end the working channel of FIG. 8A in accordance with an embodiment of the present disclosure;

FIG. 8C illustrates a cross-sectional view of a proximal end the working channel of FIG. 8A in accordance with an embodiment of the present disclosure;

FIG. 9 illustrates a detailed view of a working channel used in the method and system of providing minimally invasive percutaneous procedures in accordance with another embodiment of the present disclosure;

FIG. 10 illustrates a detailed view of a working channel used in the method and system of providing minimally invasive percutaneous procedures in accordance with another embodiment of the present disclosure with an endoscope and another tool extending therethrough;

FIG. 11A illustrates a more detailed view of a working channel used in the method and system of providing minimally invasive percutaneous procedures in accordance with another embodiment of the present disclosure;

FIG. 11B illustrates a more detailed view of a working channel used in the method and system of providing minimally invasive percutaneous procedures in accordance with another embodiment of the present disclosure;

FIG. 11C illustrates a more detailed view of a working channel used in the method and system of providing minimally invasive percutaneous procedures in accordance with another embodiment of the present disclosure;

FIG. 12A-12B illustrate an exemplary embodiment of the working channel used in the method and system of providing minimally invasive percutaneous procedures extending into a user's body;

FIG. 13 illustrates an exemplary embodiment of the working channel used in the method and system of providing minimally invasive percutaneous procedures indicating exemplary dimensions thereof; and

FIG. 14 illustrates an exemplary flow chart illustrating a method for performing percutaneous surgery in accordance with an embodiment of the present disclosure.

### DETAILED DESCRIPTION

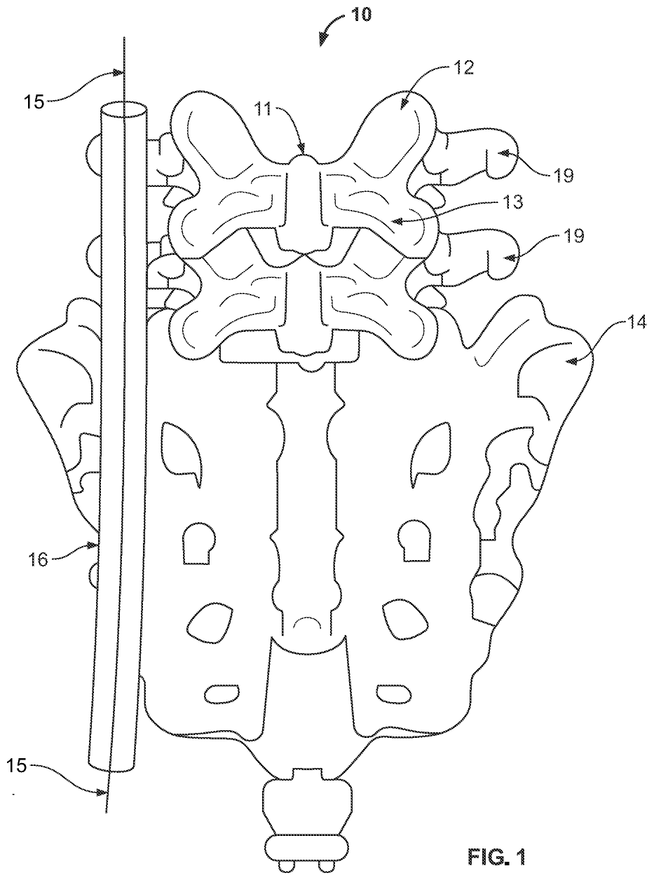

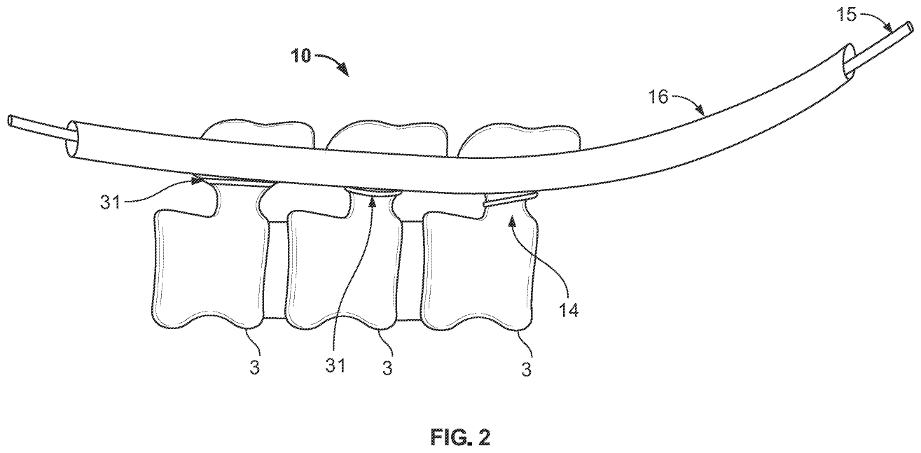

FIG. 1 is an exemplary illustration of a portion of a lumbar section of a patient's spine showing two vertebra and the ala 14. Each of the vertebrae includes the spinous process 11, the lamina 12, and the pars 13. A guide wire 15, in accordance with embodiments is illustrated with a dilator 16 shown threaded or placed over it. In embodiments, the guide wire 15 may be inserted into the patient's body following the application of an initial incision in the patient's skin and the dilator 16 may slide around the guide wire into the patient's body to expand a passage in the patient's body around the wire guide. As positioned in FIG. 1, in embodiments, the guide wire 15 and the dilator 16 are suitable for use in a spinal fusing procedure as they are positioned along the spine and extend adjacent to multiple vertebrae. FIG. 2 is a schematic diagram illustrating the dilator 16 in relation to transverse processes of the three vertebrae 30 according to an aspect of the present invention. The position of the guide wire 15 and the dilator 16 in FIGS. 1 and 2 is exemplary and would be appropriate for use in a vertebrae fusing procedure, however, they may be positioned elsewhere in and on a patient's body based on the desired procedure.

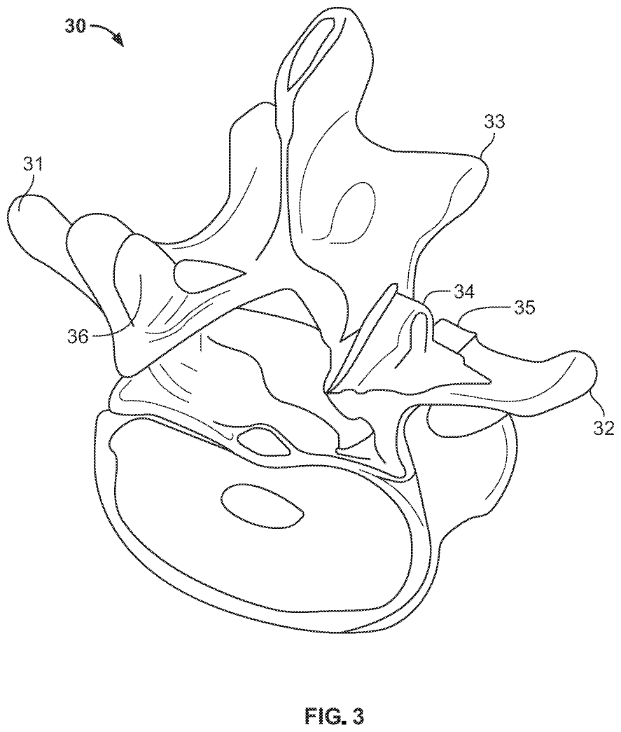

FIG. 3 illustrates an exemplary vertebra 30 of the lumbar region of a human spine showing a transverse process 32, the inferior articular process 33, the mamillary process 34, the accessory process 35 and the superior articular process 36. The vertebra 30 includes each of the foregoing structures on each side including a second transverse process 31 on the other side of vertebra 30.

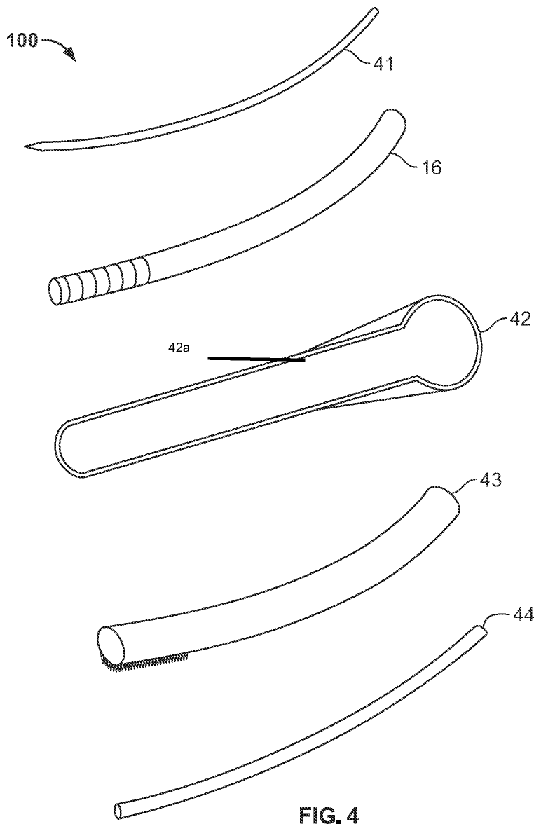

FIG. 4 shows a set or system of medical instruments 100 suitable for use in a minimally invasive percutaneous procedure according to the methodology of the present disclosure. In embodiments, a trocar 41 may have a sharp tip and is typically a solid object, like a rod. In embodiments, the trocar 41 may be curved or include a curved portion. In embodiments, the trocar 41 may be embodied as part of a cannula, such as a cannular trocar. In embodiments, in operation, after an incision is made in a user's skin, which may be made using the trocar 41 or any other suitable tool, such as a scalpel. In embodiments, the trocar 41 may be inserted into the patient's body and advanced to or through an area of interest in the patient's body after the incision is made. In embodiments, the sharp tip of the trocar 41 allows the trocar to advance through the user's body and tissue to the area of interest with relative ease and minimum trauma to surrounding tissue. In embodiments, the dilator 16 may be used to widen the channel provided by the trocar 41. In embodiments, the dilator 16 has a hollow center and slides over the in-place trocar 41 in the user's body to expand the channel made by the trocar 41. In embodiments, after the dilator 16 is positioned over the trocar 41, the trocar may be removed through the dilator. In embodiments, as noted above, a guide wire 15 may be advanced to or through the area of interest in the patient's body and the dilator 16 may slide over the guide wire. In embodiments, the guide wire 15 may be used without the trocar 41 and may be withdrawn from the dilator 16 after the dilator is positioned.

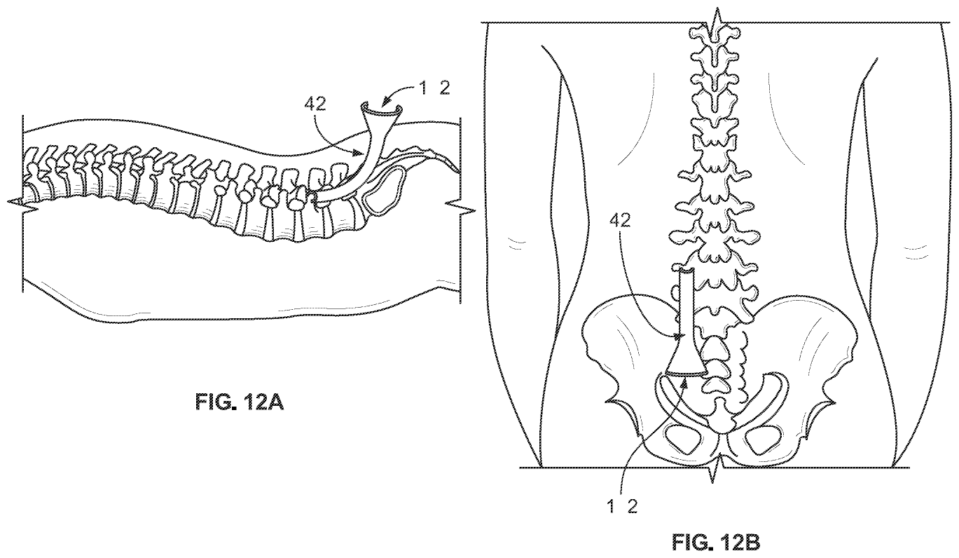

In embodiments, a working channel 42 may be positioned around the dilator 16 to allow other instruments, such as endoscopes 122 (see FIG. 12A, for example) to be provided into the patient's body to facilitate viewing the condition of tissue or the like in the patient's body and well as to allow access to the patient's body in a minimally invasive manner. The endoscope 122, once in the channel 42, may be used to view any component of the spine, whether the lamina, the transverse processes, the facet joints, the discs or the vertebral bodies depending on a position of insertion along the spine. The endoscope 122 may be used to view other parts of the body as well, in embodiments, for example where the channel is used for other procedures. In this manner, various procedures, including lumbar laminectomies, for example, may be performed in any part of the spine, including the lumbar, thoracic or cervical areas. In embodiments, discectomies may also be performed using the endoscope 122 and channel 42 as well an any other tools via the channel. In embodiments, the channel 42 allows a variety of procedures to be performed endoscopically, that is, with the aid of an endoscope 122 that can be moved within the channel to access and view portions of the patient's body along the length of the channel with a minimally invasive approach. In embodiments, as generally noted above, insertion of the working channel 42 will follow the insertion of the dilator 16 and the dilator will be removed to facilitate positioning of tools, such as the endoscope 122 in the channel 42.

In embodiments, a decorticator such as rasp 43, a pushing instrument 44, a syringe for providing medication or other substances to a region of interest in the patient's body may be provided in the patient's body via the working channel 42. In embodiments, the working channel 42 includes an inner wall 42b and an outer wall 42c surrounding the inner wall. In embodiments, the at least one side opening 42a extends between the inner wall 42b and the outer wall 42c and along the length of the working channel 42 to provide access to the patient's body along the length of the working channel. In embodiments, tissue, bone, or specimens may be removed from the region of interest at any point along the length of the channel 42 and removed from the body via the working channel 42. In embodiments, the rasp 43 is typically solid and includes a serrated surface or distal edge. In embodiments, the pusher 44 may be generally solid, or at least include a forward directed solid surface that may be used to push medication, a liquid or other substance, such as a bone matrix or bone fusion substance to the region of interest inside the user's body through the working channel 42. In embodiments, the syringe may be used to provide medication or other materials at any point along the length of the working channel 42.

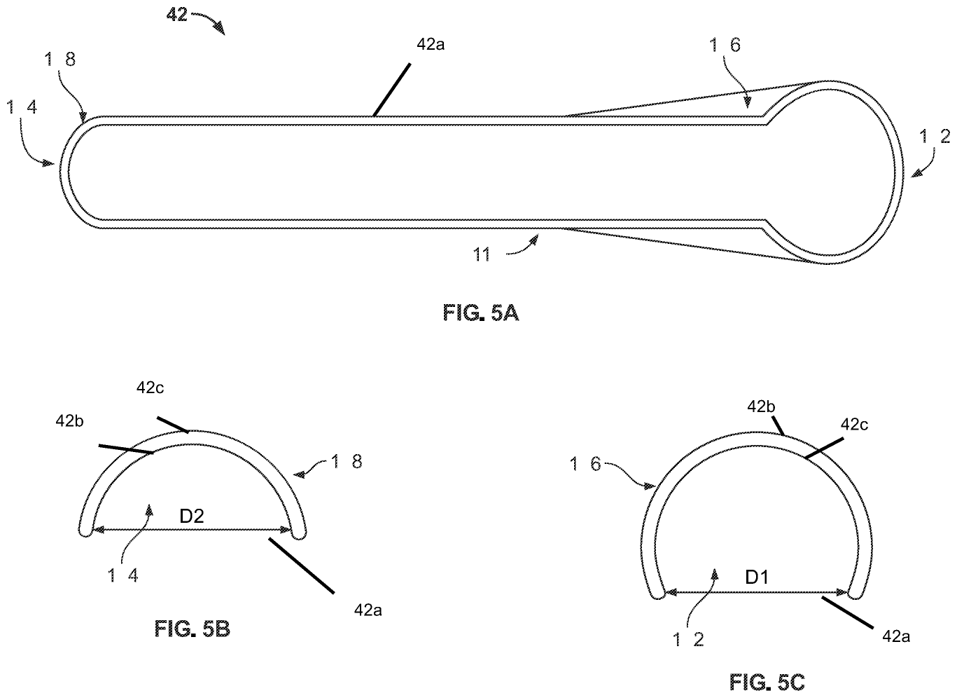

In embodiments, the working channel 42 may be made of any suitable substantially rigid material including but not limited to surgical steel or other sterilizable metal or plastic. As noted above, the working channel 42 includes a side opening 42a extending between the inner wall 42b and the outer wall 42c and along the length of the channel 42 to allow access to the patient's body along the length of the channel. In embodiments, the working channel 42 may have a C-shaped cross-section with the open portion of the C corresponding to the side opening 42a as can be seen in FIGS. 5B and 5C, for example. In embodiments, a different cross-section may be provided as long as the side opening 42a allows access along the length of the working channel 42. In embodiments, the working channel 42 may include a proximal, wide opening 102 with a diameter D1 (see FIGS. 5A and 5C, for example) on a first end thereof that is larger than a diameter D2 (see FIG. 5B, for example) of a distal, narrow opening 104 on the second, opposite end thereof. In embodiments, the diameter D1 of the proximal wide opening 102 may range from 2-4 cm. In embodiments, the diameter D2 of the distal opening 104 may be 1-2 cm. In embodiments, the wide proximal opening 102 accommodates the introduction of additional instruments as well as the endoscope 122 as needed, for example the decorticator or a drill to allow for endoscopic procedures along the length of the channel.

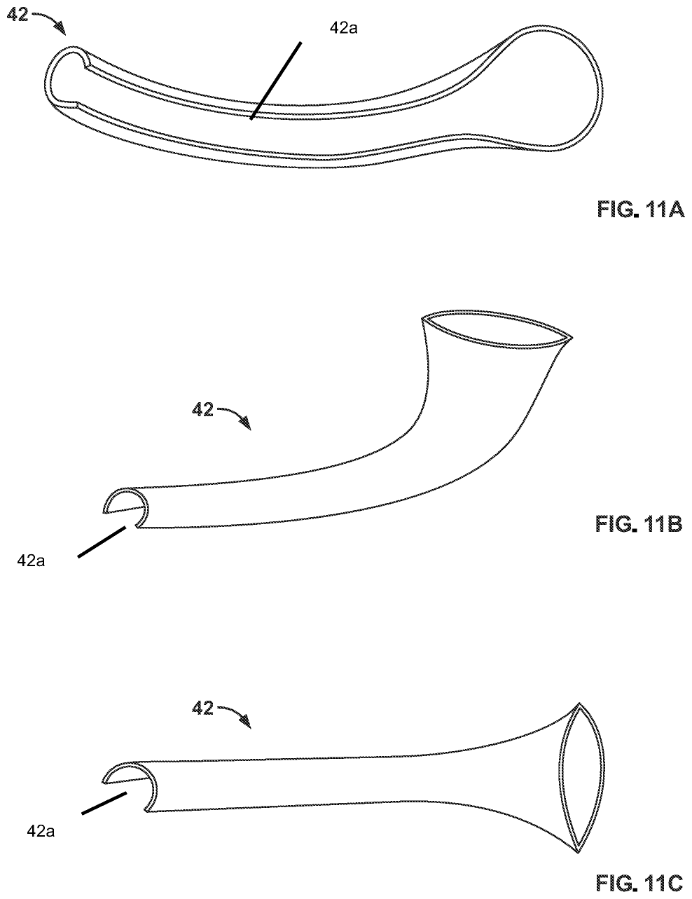

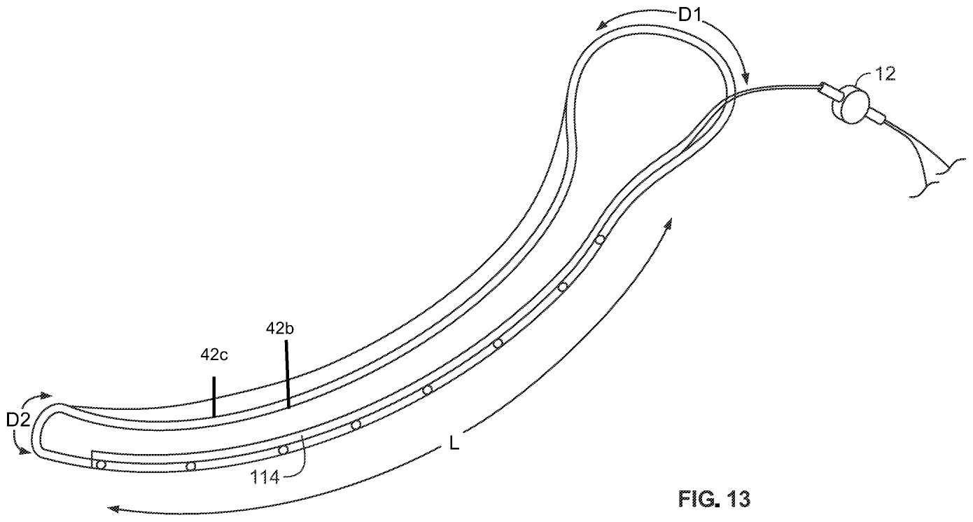

In embodiments, the channel 42 may have a curved shape relative to its length as can be generally seen in FIGS. 11A, 11B and 11C, 12A and 13, for example. The curve of the working channel 42 may vary depending on the application for which it is being used. For example, a working channel 42 appropriate for use in vertebrae fusing procedure referenced with respect to FIG. 2, may be different than the curve of a working channel 42 that is used in another procedure. In embodiments, the curve of the working channel 42 appropriate for use in spinal fusion of the portion of the spine shown in FIG. 2 may be different than the curve of a working channel 42 that may be used for spinal fusion on another portion of the spine. In embodiments, the working channel may include more than one curve or curved portion. In embodiments, the length L (see FIG. 13, for example) of the working channel 42 may range from 16-24 cm. In embodiments, the dimensions of the working channel 42 may vary such that any clinically relevant dimensions may be used provided that the wide opening 102 is wider than the narrow opening 104. In embodiments, a thickness of the wall of the working channel 42 may vary provided that it remains sufficiently rigid to maintain its shape when it is in place in the patient's body. In embodiments, the working channel 42 is substantially rigid to retain its shape when inserted in the patient's body to maintain access to the area of interest.

In embodiments, the wide proximal opening 102 remains outside of the patient's skin, as can be seen in FIGS. 12A and 12B, for example, and allows for easier insertion and access for and manipulation of surgical instrumentation, including endoscope 122 and/or suction or any other instrument 124, such as a drill, inside the patient's body via the working channel 42 by the clinician or a robotic system. That is, the wider opening 102 on the proximal end of the working channel 42 provides room for a user to manipulate tools or accessories, including the endoscope 122, rasp 43, trocar 41, pusher 44, syringe or other instrument 124 to pass through the proximal opening 102 into the patient's body while the narrow distal opening 104 provided at the distal end minimizes disruption to tissue inside the user's body to an area near the area of interest. In embodiments, as noted above, the side opening 42a extends along the length of the working channel 42a such that the tools or accessories may be used along the length of the working channel 42 at various positioned in the patient's body.

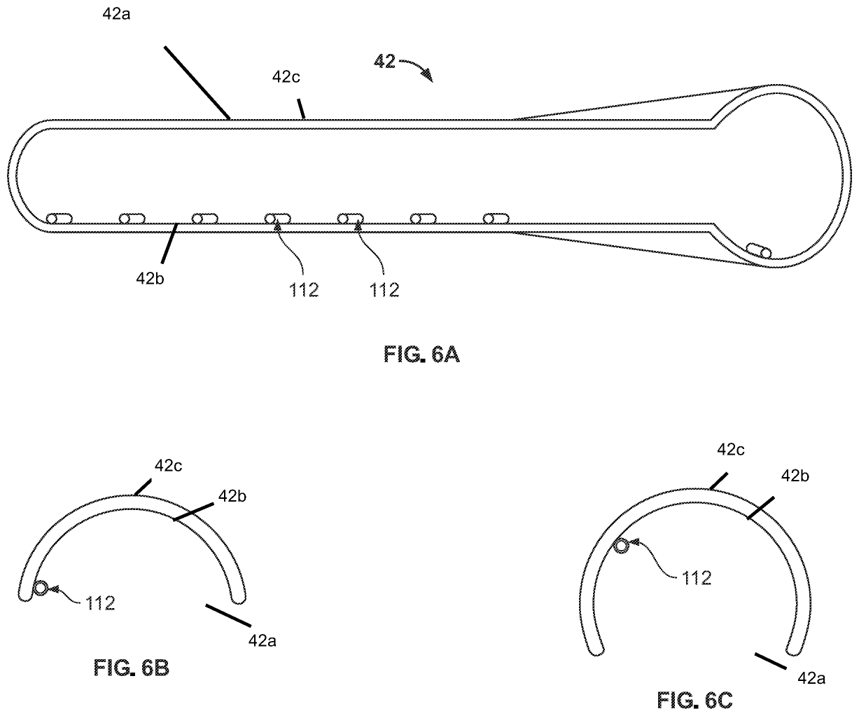

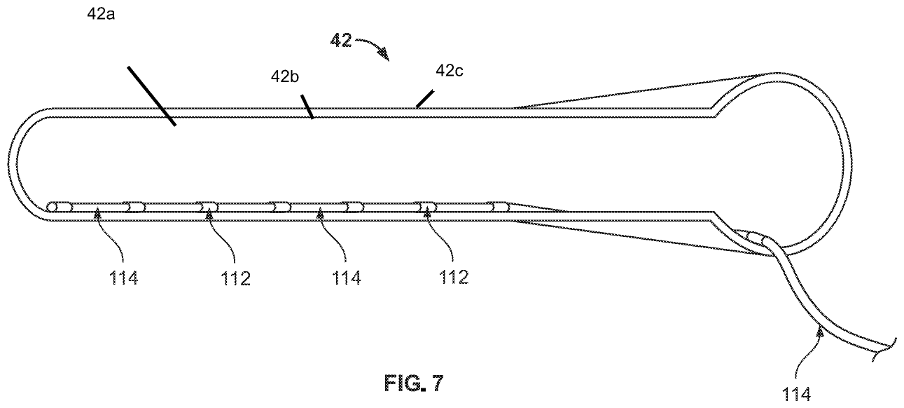

In embodiments, the working channel 42 may include suction mounting elements 112 (see FIGS. 6A, 6B and 6C) configured to receive and secure standard suction tubing 114 to the working channel 42 as can be seen in FIG. 7 such that suction may be provided in addition to the tools discussed above through the channel 42. In embodiments, the suction mounting elements 112 may be rings that the suction tubing 114 is threaded through, as illustrated in FIG. 7. In embodiments, the suction mounting elements 112 may be clips or open-mounts or any other suitable structures for holding the suction tubing 114 in place on working channel 42 during a procedure. In embodiments, the working channel 42 may include more or fewer mounting elements 112. In embodiments, the suction mounting elements 112 may be provided on an inner wall 42b of the working channel 42.

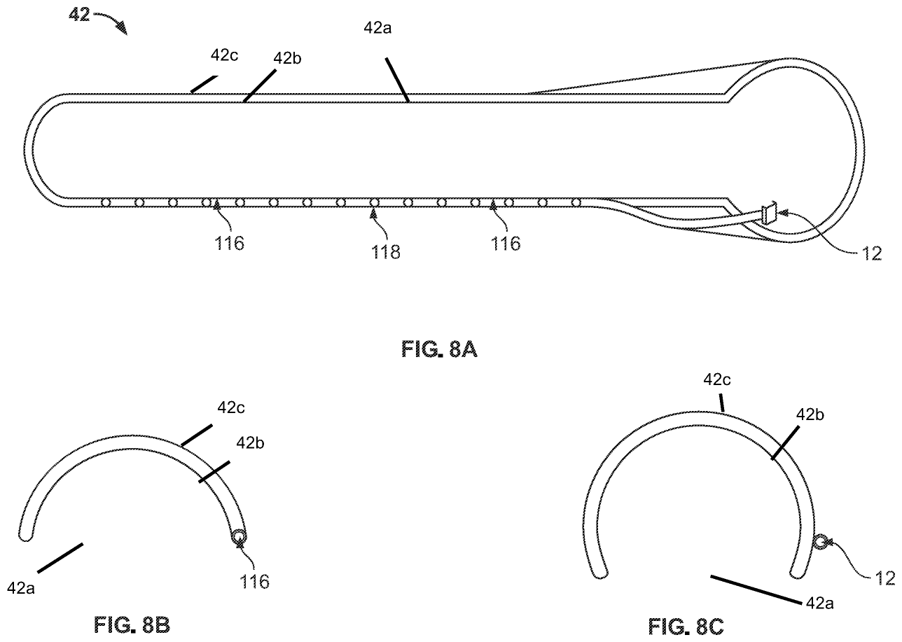

In embodiments, the working channel 42 may include an integrated suction element 116 (see FIG. 8A, for example). In embodiments, the integrated suction element 116 may include multiple suction openings 118 incorporated therein and attached to a standard suction source outside of the patient's body via a suction port or connector 120. In embodiments, fewer or additional suction openings 118 may be provided in the integrated suction element 116. In embodiments, the integrated suction element 116 may be built either partially or fully into the wall of the working channel 42 between the inner wall 42b and outer wall 42c, for example, as can be seen in FIGS. 8A and 8B, for example, and may be substantially flush with the inner wall of the channel 42. In embodiments, the integrated suction element 116 or a portion thereof may be integrated onto a wall surface of the channel 42 as illustrated in FIG. 8C, for example. In embodiments, the suction element 116 may be integrated into the wall of the working channel 42. In embodiments, the port or other connector 120 may connect the integrated suction element 116 to an external suction source which is typically positioned outside of the user's body. In embodiments, the port or other connector 120 may be integrated into the wall of the working channel 42. In embodiments, the port or other connector 120 may be connected to a tube or hose connected to a suction source or supply.

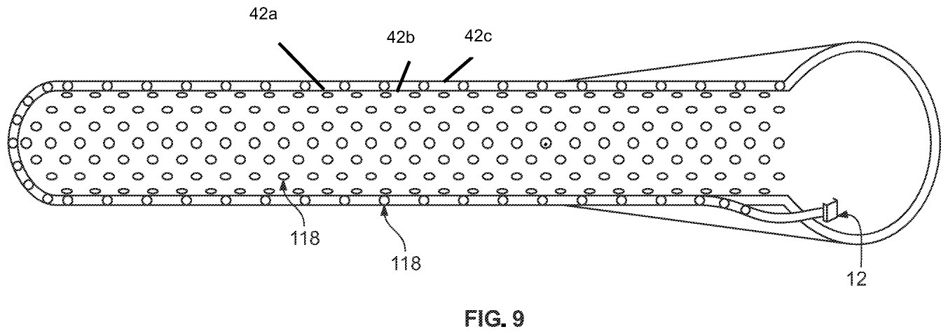

In embodiments, the suction openings 118 may be formed across the entire, or substantially the entire inner surface the inner wall 42b of the working channel 42 as illustrated in FIG. 9, for example. In embodiments, the suction openings 118 may be provided across the exterior surface of the outer wall 42c of the working channel 42, if desired. In embodiments, the integrated suction element 116 may include multiple portions formed in the wall of the working channel 42 between the inner wall 42b and the outer wall 42c and in fluid communication with the plurality of suction openings 118. In embodiments, the wall of the working channel 42 between the inner wall 42b and the outer wall 42c may be substantially hollow such that all of the suction openings 118 are in fluid communication with each other. In embodiments, fewer or additional suction openings 118 may be used. In embodiments, a single suction opening 118 may be provided at or near the distal narrow opening 104 of the channel 42, or elsewhere along the length of the working channel 42.

In embodiments, the working channel 42 may be curved along its length L as noted above, however, the working channel 42 may have a variety of different shapes based on clinical needs. In embodiments, the working channel 42 may include curves in different directions, for example.

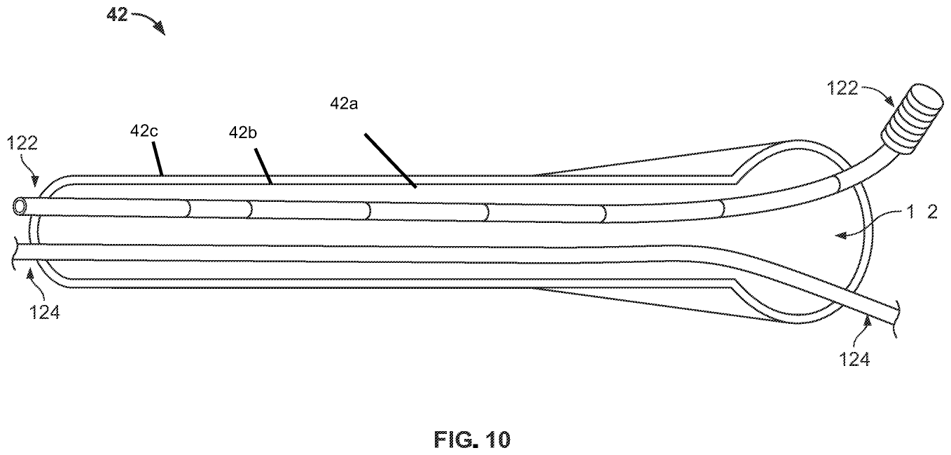

In embodiments, the working channel 42 may be configured to receive additional tools or accessories that may be used in the procedure including the endoscope 122 and an additional tool 124, such as a drill. As noted above, the suction tube 114 may extend through the channel 42 or the integrated suction element 116 may be integrated into the wall of the channel 42. In an embodiment, such as that illustrated in FIG. 10, for example, the wide opening 102 allows additional room for inserting and manipulating the endoscope 122 and any other tools 124 from outside of the patient's body and provides access to the interior of the patient's body through the working channel 42. As can be seen in FIGS. 12A and 12B, the wide opening 102 extends outside the user's body to make insertion of the tools easier. While FIGS. 12A and 12B illustrate positioning of the working channel 42 in the patient's back, for example, it may be positioned elsewhere to allow for minimally invasive percutaneous procedures at virtually any desired position on a patient's body while allowing for the insertion and use of various tools and accessories such that a wide variety of procedures may be performed.

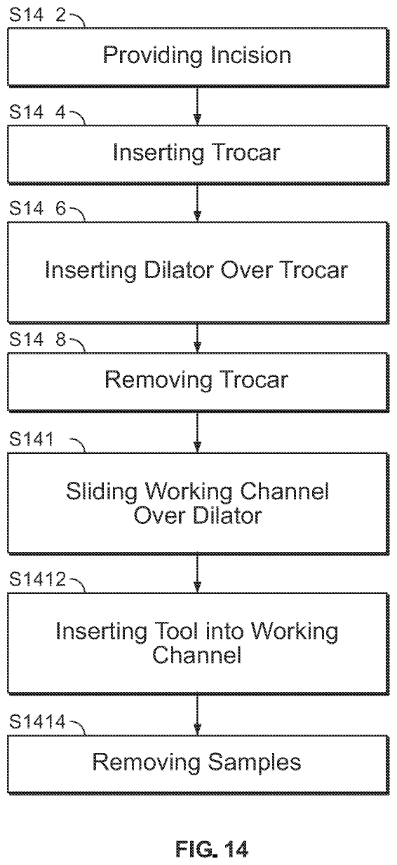

FIG. 14 illustrates an exemplary flow chart illustrating a method for performing percutaneous surgery using the working channel 42. In use, a user will first make an incision in a patient's skin, for example, at step S1402, which may be made using a scalpel or other suitable instrument. In embodiments, as noted above, the incision may be made using the trocar 41. Thereafter, in embodiments, the trocar 41 and/or a guide wire 15 may be inserted into the user's body and advanced to or through the area of interest in the user's body at step S1404. Thereafter, in embodiments, the dilator 16 may slide over the trocar 41 or guide wire 15 to widen the path around the trocar or wire inside the patient's body at step S1406. Thereafter, the trocar 41 or guide wire 15 may be removed and withdrawn from the patient's body through the dilator at step S1408. In embodiments, the working channel 42 may slide over the dilator 16 into the user's body such that the narrow opening 104 is positioned in the patient's body substantially at or beyond the area of interest and the wide opening 102 is positioned outside the user's skin at step S1410. The dilator 16 may be withdrawn and removed leaving the working channel 42 in place with the narrow opening 104 positioned in the user's body under the user's skin and the wide opening 102 extending outside the user's body outside the user's skin. In embodiments, a suction tube 114 may be attached to the working channel 42 via the mounting elements 112, for example. In embodiments, as noted above, the working channel 42 may include an integrated suction portion 116 that may be connected to a suction source via the connector 120. In embodiments, the suction tube 14 may be provided in the working channel and used in the procedure without use of he mounting elements. In embodiments, an endoscope 122, rasp 43, pusher 44, syringe or other tool, such as a drill, for example, may be inserted into the wide opening 102 and extend through the working channel 42 into the patient's body toward the narrow opening 104 step S1412. In embodiments, other tools 124, including the rasp 43 and pusher 44 may also be inserted into the wide opening 102 to extend through the working channel 42 to the narrow opening 104. In embodiments, tissue, bone, or other specimens from the patient's body may be withdrawn via the working channel 42 at step S1414.

While particular embodiments of the present disclosure have been shown and described in detail, it would be obvious to those skilled in the art that various modifications and improvements thereon may be made without departing from the spirit and scope of the disclosure.

What is claimed is:

1. A system for performing percutaneous procedures comprises:
a working channel,
the working channel including:
an inner wall;
an outer wall surrounding the inner wall;
a side opening formed between the inner wall and the outer wall along a length of the working channel;
a first end having a first width;
a second end having a second width, wherein the second width is less than the first width; and
at least one tool configured to be received in the working channel.

2. The system of claim 1, further comprising at least one fastener connected to the working channel, wherein the at least one fastener is configured to receive a suction hose connected to a suction source.

3. The system of claim 1, further comprising an integral suction element formed in at least one of the inner wall and the outer wall of the working channel.

4. The system of claim 3, wherein the inner wall includes at least one suction opening formed therein and in fluid communication with the integral suction element.

5. The system of claim 4, further comprising a suction connector provided in at least one of the inner wall and the outer wall and configured to provide a connection between the integral suction element and a suction supply.

6. The system of claim 1, wherein the at least one tool comprises a trocar configured to make an incision in a patient's skin and extend into the patient's body.

7. The system of claim 6, wherein the working channel is configured to enter the user's skin with the first end positioned under the user's skin and the second end extending outside of the user's skin.

8. The system of claim 7, wherein the trocar is configured to be received in the working channel and withdrawn therefrom after the trocar reaches a desired position in the patient's body.

9. The system of claim 7, further comprising a second tool configured to be received in the inner cavity of the working channel.

10. The system of claim 9, wherein the second tool comprises a dilator configured to expand a path made by the trocar through the user's body and configured to be removed from the working channel after extending to the point of interest in the user's body.

11. The system of claim 9, wherein the second tool comprises an endoscope.

12. The system of claim 9, wherein the second tool comprises a syringe.

13. The system of claim 9, wherein the second tool comprises a decorticator.

14. A working channel comprising:
an inner wall;
an outer wall surrounding the inner wall;
a side opening formed between the inner wall and the outer wall along a length of the working channel;
a first end having a first width; and
a second end having a second width, wherein the second width is less than the first width.

15. The working channel of claim 14, further comprising at least one fastener connected to the working channel, wherein the at least one fastener is configured to receive a suction hose connected to a suction source.

16. The working channel of claim 14, further comprising an integral suction element formed in at least one of the inner wall and the outer wall.

17. The working channel of claim 16, further comprising at least one suction opening formed in the inner wall and in fluid communication with the integral suction element.

18. The working channel of claim 16, further comprising a plurality of suction openings formed in the inner wall and in fluid communication with the integral suction element.

19. The working channel of claim 16, further comprising at least suction connector configured to provide a fluid connection between the integral suction element and a suction supply.

20. The working channel of claim 16, further comprising a second integral suction element formed in a least one of the inner wall and the outer wall.
